(19) United States
(12) Patent Application Publication
(10) Pub. No.: US 2022/0233356 A1
(43) Pub. Date: Jul. 28, 2022

(54) FACE SHIELD APPARATUS

(71) Applicant: Ashley Bezamat, Los Angeles, CA (US)

(72) Inventor: Ashley Bezamat, Los Angeles, CA (US)

(21) Appl. No.: 17/588,090

(22) Filed: Jan. 28, 2022

Related U.S. Application Data
(60) Provisional application No. 63/142,607, filed on Jan. 28, 2021.

(51) Int. Cl. A61F 9/02; A41D 13/11

(52) U.S. Cl. CPC A61F 9/027; A61F 9/029; A41D 13/11

(57) ABSTRACT

A pair of opposed locking clasps couples a temple of a frame to a top bar of the frame. The pair of opposed locking clasps are comprised of a first locking clasp, a second locking clasp, and a hinge. The first locking clasp is comprised of a first base, a nub disposed proximate to a first end of the first base, and a protrusion disposed proximate to a second end of the first base. The second locking clasp is comprised of a second body and an indent that substantially corresponds to a shape of the protrusion, the protrusion being disposed in the indent to lock the first locking clasp to the second locking clasp. The hinge couples the first locking clasp to the second locking clasp.

### CROSS-REFERENCE TO RELATED APPLICATION

This application claims priority from U.S. Provisional Patent Application Ser. No. 63/142,607 filed on Jan. 28, 2021, entitled “FACE SHIELD APPARATUS”, the entire disclosure of which is hereby incorporated by reference in its entirety.

### BACKGROUND OF THE DISCLOSURE

### 1. Field of the Disclosure

The disclosure relates in general to personal protection equipment, and more particularly, to a face shield apparatus.

### 2. Background Art

While face shields have been known in the art of personal protection equipment, with the recent pandemic, their use has become substantially more widespread. Additionally, such use has expanded outside of the traditional areas of use (high splatter environments in assembly, medicine and the like). In many instances, to achieve an additional level of protection, such face shields are worn by users where interactions with other people (customers, patients, persons, colleagues, patrons or the like) can be common and/or reoccurring.

With the increasing use and in increasingly different venues, problems have arisen with traditional face shields. For example, these shields can be worn for longer durations that initially designed. As a result, many prior art shields become uncomfortable to use for extended periods of time. Furthermore, with extended use, use with glasses (or even sun glasses) is desirable and not always possible with prior designs. Still further, some prior art designs sealingly engage with the forehead of a user which is not only uncomfortable but which creates a sealed environment within the face shield space. Still other prior art shields that have separate frames can be very difficult to attach to the frame or have couplings that do not provide a sturdy and durable coupling.

### SUMMARY OF THE DISCLOSURE

The disclosure is directed to a pair of opposed locking clasps to couple a temple of a frame to a top bar of the frame. The pair of opposed locking clasps are comprised of a first locking clasp, a second locking clasp, and a hinge. The first locking clasp is comprised of a first base, a nub disposed proximate to a first end of the first base, and a protrusion disposed proximate to a second end of the first base. The second locking clasp is comprised of a second body and an indent that substantially corresponds to a shape of the protrusion, the protrusion being disposed in the indent to lock the first locking clasp to the second locking clasp. The hinge couples the first locking clasp to the second locking clasp.

In at least one configuration of the pair of opposed locking clasps, the protrusion includes a non-uniform outer perimeter so as to preclude rotative movement therearound.

In at least one configuration of the pair of opposed locking clasps, the protrusion is an elongated racetrack oval.

In at least one configuration of the pair of opposed locking clasps, the protrusion includes a forward flange disposed on a forward end of the protrusion and extends beyond an outer perimeter of the protrusion.

In at least one configuration of the pair of opposed locking clasps, the hinge is a live hinge.

In at least one configuration of the pair of opposed locking clasps, the nub is circular in shape.

In at least one configuration of the pair of opposed locking clasps, the pair of opposed locking clasps comprise a flexible polymer.

In at least one configuration of the pair of opposed locking clasps, the frame is molded from a polymer.

In at least one configuration of the pair of opposed locking clasps, the polymer is at least one of TR90 and a nylon.

In at least one configuration of the pair of opposed locking clasps, a face shield apparatus is comprised of the pair of opposed locking clasps, and the face shield apparatus further comprises a shield that is coupled to the fame via the pair of opposed locking clasps.

In at least one configuration of the pair of opposed locking clasps, the shield is a full-face shield.

In at least one configuration of the pair of opposed locking clasps, the shield wraps around a face of a wearer of the face shield apparatus.

The disclosure is also directed to a face shield apparatus that is comprised of a pair of opposed locking clasps to couple a temple of a frame to a top bar of the frame, and a shield. The pair of opposed locking clasps are comprised of a first locking clasp, a second locking clasp, and a hinge. The first locking clasp is comprised of a first base, a nub disposed proximate to a first end of the first base, and a protrusion disposed proximate to a second end of the first base. The second locking clasp is comprised of a second body and an indent that substantially corresponds to a shape of the protrusion, the protrusion being disposed in the indent to lock the first locking clasp to the second locking clasp. The hinge couples the first locking clasp to the second locking clasp. The shield is coupled to the fame via the pair of opposed locking clasps.

In at least one configuration of the face shield apparatus, the protrusion includes a non-uniform outer perimeter so as to preclude rotative movement therearound.

In at least one configuration, the protrusion is an elongated racetrack oval.

In at least one configuration, the protrusion includes a forward flange disposed on a forward end of the protrusion and extends beyond an outer perimeter of the protrusion.

In at least one configuration, the hinge is a live hinge.

In at least one configuration, the nub is circular in shape.

In at least one configuration, the pair of opposed locking clasps comprise a flexible polymer.

In at least one configuration, the shield is a full-face shield.

### BRIEF DESCRIPTION OF THE DRAWINGS

The disclosure will now be described with reference to the drawings wherein:

FIG. 1 of the drawings is a top perspective view of the face shield apparatus of the present disclosure;

FIG. 2 of the drawings is a bottom perspective view of the face shield apparatus of the present disclosure;

FIG. 3 of the drawings is a front elevational view of the face shield apparatus of the present disclosure;

FIG. 4 of the drawings is first side elevational view of the face shield apparatus of the present disclosure;

FIG. 5 of the drawings is a second side elevational view of the face shield apparatus of the present disclosure;

FIG. 6 of the drawings is a top plan view of the face shield apparatus of the present disclosure;

FIG. 7 of the drawings is a bottom plan view of the face shield apparatus of the present disclosure;

FIG. 8 of the drawings is a partial perspective view of the frame with the locking clasp installed on the first temple 24;

FIG. 9 of the drawings is a partial perspective view of the frame showing the shield attachment member of the first temple 24;

FIG. 10 of the drawings is an inside perspective view of the locking clasp of the present disclosure;

FIG. 11 of the drawings is an outside perspective view of the locking clasp of the present disclosure;

FIG. 12 of the drawings is a top plan view of the locking clasp of the present disclosure;

FIG. 13 of the drawings is a bottom plan view of the locking clasp of the present disclosure;

FIG. 14 of the drawings is a first side view of the locking clasp of the present disclosure;

FIG. 15 of the drawings is a second side view of the locking clasp of the present disclosure;

FIG. 16 of the drawings is an end view of the locking clasp of the present disclosure;

FIG. 17 of the drawings is a first perspective view of a standalone shield attachment member with locking clasp, in an open, and partially overlying position;

FIG. 18 of the drawings is a second perspective view of the standalone shield attachment member with locking clasp, in an open, and partially overlying position;

FIG. 19 of the drawings is a first side elevational view of the standalone shield attachment member with locking clasp, in an open, and partially overlying position;

FIG. 20 of the drawings is a bottom plan view of the standalone shield attachment member with locking clasp, in an open, and partially overlying position;

FIG. 21 of the drawings is a front elevational view of the standalone shield attachment member with locking clasp, in an open, and partially overlying position;

FIG. 22 of the drawings is a top plan view of the standalone shield attachment member with locking clasp, in an open, and partially overlying position;

FIG. 23 of the drawings is a second side elevational view of the standalone shield attachment member with locking clasp, in an open, and partially overlying position;

FIG. 24 of the drawings is a first perspective view of a standalone shield attachment member with locking clasp, in a closed position;

FIG. 25 of the drawings is a second perspective view of the standalone shield attachment member with locking clasp, in a closed position;

FIG. 26 of the drawings is a first side elevational view of the standalone shield attachment member with locking clasp, in a closed position;

FIG. 27 of the drawings is a bottom plan view of the standalone shield attachment member with locking clasp, in a closed position;

FIG. 28 of the drawings is a front elevational view of the standalone shield attachment member with locking clasp, in a closed position;

FIG. 29 of the drawings is a top plan view of the standalone shield attachment member with locking clasp, in a closed position;

FIG. 30 of the drawings is a second side elevational view of the standalone shield attachment member with locking clasp, in a closed position;

FIG. 31 of the drawings is a first perspective view of a standalone shield attachment member with locking clasp, in a flattened open position;

FIG. 32 of the drawings is a second perspective view of the standalone shield attachment member with locking clasp, in a flattened open position;

FIG. 33 of the drawings is a first side elevational view of the standalone shield attachment member with locking clasp, in a flattened open position;

FIG. 34 of the drawings is a bottom plan view of the standalone shield attachment member with locking clasp, in a flattened open position;

FIG. 35 of the drawings is a front elevational view of the standalone shield attachment member with locking clasp, in a flattened open position;

FIG. 36 of the drawings is a top plan view of the standalone shield attachment member with locking clasp, in a flattened open position; and

FIG. 37 of the drawings is a second side elevational view of the standalone shield attachment member with locking clasp, in a flattened open position.

### DETAILED DESCRIPTION OF THE DISCLOSURE

While this disclosure is susceptible of embodiment in many different forms, there is shown in the drawings and described herein in detail a specific embodiment(s) with the understanding that the present disclosure is to be considered as an exemplification and is not intended to be limited to the embodiment(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

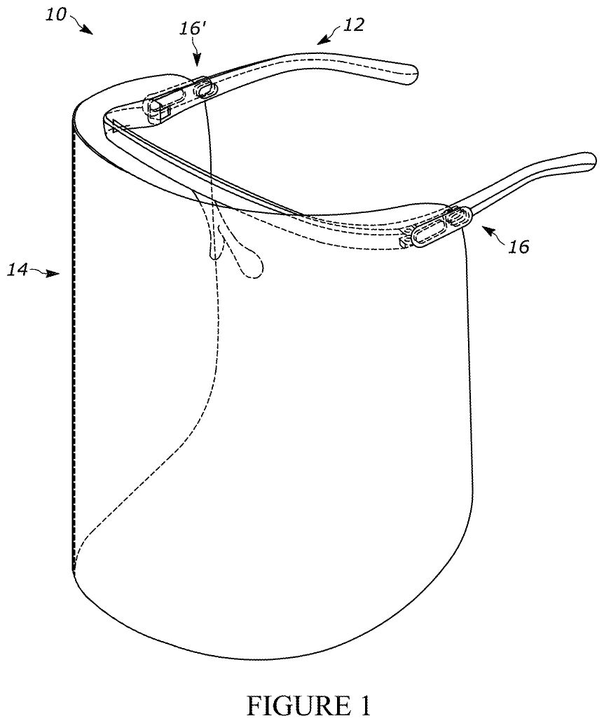

Referring now to the drawings and in particular to FIGS. 1 and 7, the face shield apparatus is shown generally at 10. The face shield apparatus 10 includes frame 12, shield 14 and locking clasps 16 and 16′. The face shield apparatus 10 can be used alone, or in conjunction with other forms of personal protection equipment (PPE). Additionally, the face shield apparatus 10 is configured for use along with glasses while minimizing the interference therewith.

The frame 12 of the face shield apparatus 10 includes top bar 20, bridge 22 and first and second temples 24 and 24′. While not required, in the configuration shown, the top bar 20 and the bridge 22 are integrally molded, and the frame 12 is preferably molded from a polymer, such as TR90 available from EMS-Grivory of Domat, Switzerland, a nylon or another polymer. Such a polymer allows for extensive flexibility and elastic deformation without cracking or deforming. Other materials that are contemplated include a composite material (fiberglass or carbon fiber based), a metal such as stainless steel and titanium among other metals and alloys thereof. Of course, the selection of material is not to be deemed limiting, but, rather is solely intended to be exemplary.

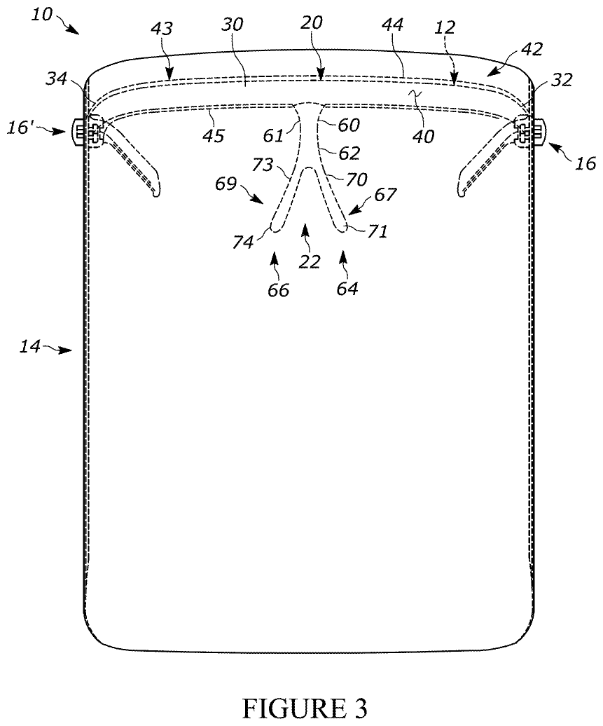

With reference to FIGS. 3 through 6, the frame 12 is defined by front portion 30, first end portion 32 and second end portion 34. The front portion 30 extends, preferably over the forehead of the user, and includes outward surface 40, inward surface 41, first side 42, second side 43, upper edge 44 and lower edge 45. The outward surface 40 is preferably substantially planar, while some curvature is contemplated as well toward the opposing first and second ends. In another configuration, the frame 12 may extend all the way around the user (in the form of a headband or the like) and may comprise a single component.

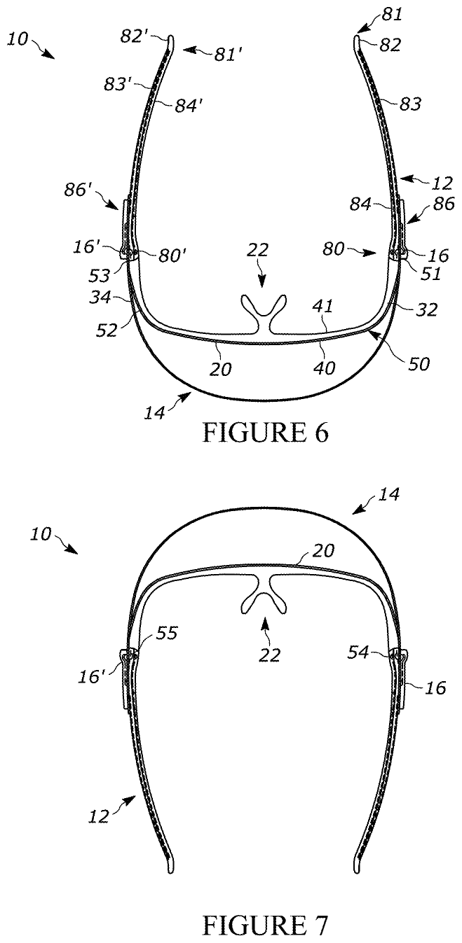

With continued reference to FIGS. 3 through 6, the first end portion 32 has a front end 50 and a temple end 51 (FIG. 6). The front end 50 extends from the first side 42 of the front portion 30 to terminate at temple end 51. The first end portion 32 has an arcuate configuration so as to form a bend that is close to 90° (i.e., 90°+/−10°). Additionally, the first end portion 32 is angled in a downward direction between the front end 50 and the temple end so that the front portion 30 rests over the forehead while the temple end 51 rests lower (and, in some configurations, substantially even with the upper end of the ear so that the temples 24 and 24′ are substantially horizontal). In some configurations, the downward angle of the front-end portion (angle α) may be approximately 19° (more preferably between 5° and 45°). Similarly, the second end portion 34 has front end 52 that extends from the second side 43 of the front portion 30 and terminates at temple end 53. The second end portion 34 is a mirror image of the first end portion 32 taken about a line that bisects the front portion 30 of the top bar 20.

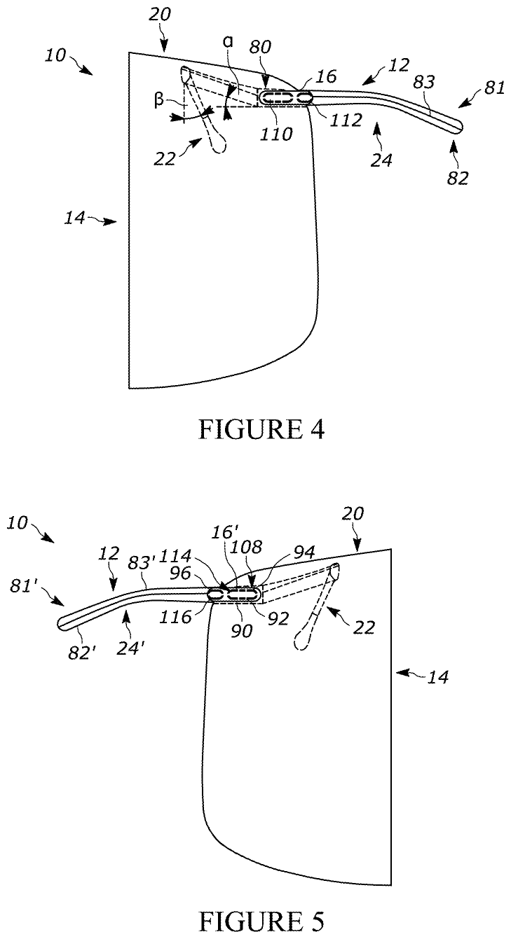

With reference to FIGS. 3 and 4, the bridge 22 includes central portion 60, first nose pad arm 64 and second nose pad arm 66. The central portion 60 depends from the front portion 30 and includes upper end 61 and lower end 62. The first nose pad arm 64 has a proximal end 70 that extends from the lower end 62 of the central portion 60 and terminates at distal end 71. A first nose pad 67 overlies a portion of the first nose pad arm 64. The second nose pad arm 66 has a proximal end 73 that extends from the lower end 62 of the central portion 60 and terminates at a distal end 74. A second nose pad 69 overlies a portion of the second nose pad arm 66. The central portion 60 and the first and second nose pad arms 64 and 66 are inclined downward and rearwardly at an angle (angle (3) of approximately 25° (i.e., between 5° and 45°) relative to a vertical line depending from the outward surface 40 of the front portion 30. Such an angle facilitates the directing of the front portion 30 away from the forehead of the user. Additionally, such an angle provides for the bridge 22 to engage proximate an upper end of the nose allowing for the user to also utilize glasses that may be positioned outboard of the bridge 22 on the nose of the user.

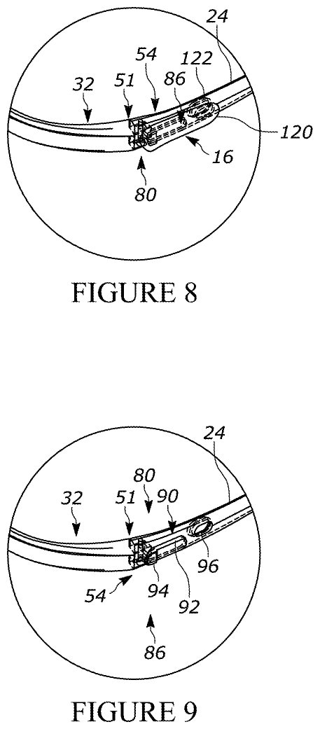

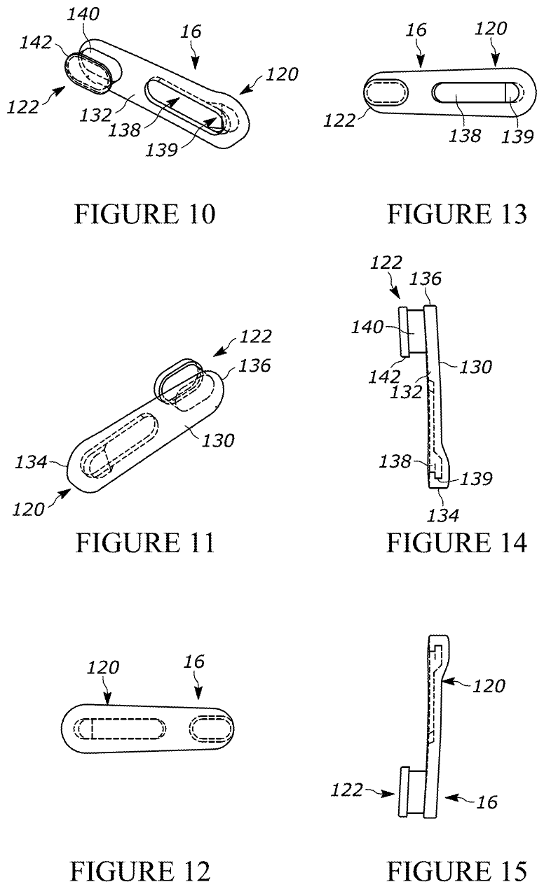

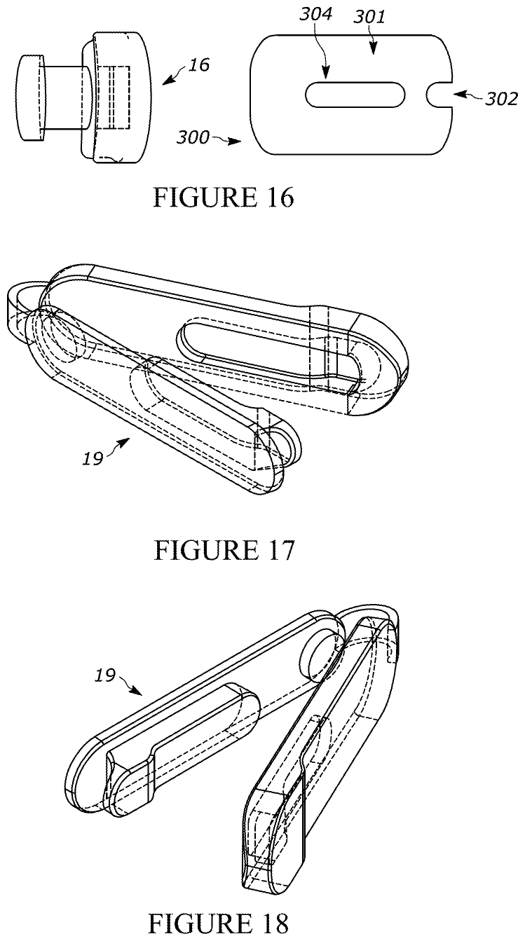

With reference to FIGS. 4 through 7 (and with selective additional reference to FIGS. 8 and 9) The first temple 24 extends from the first end portion 32 and is hinged thereto through hinge 54 which may include a hinge pin or which may be formed from mating components of the first end portion 32 and the first temple 24. The first temple 24 includes proximal end 80 (which is proximate the hinge 54) and distal end 81. The distal end 81 terminates at the temple tip 82. The first temple 24 includes outer surface 83 and inner surface 84. In the configuration shown, the first temple 24 is substantially planar with a downward portion toward the distal end 81 to extend about the ear of a user.

With reference to FIG. 9, the shield attachment member 86 is disposed near the proximal end 80 of the first temple 24. The shield attachment member 86 includes a protrusion 90 which extends outwardly from the outer surface 83 of the first temple 24. The protrusion 90 has a non-uniform outer perimeter 92 so as to preclude rotative movement therearound. To enhance the rigidity of the interface between the protrusion 90, 90′ and a bore of the shield 110, 114 (as will be described below), the protrusion 90 is formed as an elongated racetrack oval configuration with the elongation extending along the outer surface between the proximal end and the distal end 81 of the first temple 24. Other configurations and shapes are contemplated, both shapes that are uniform and non-uniform, and/or arbitrary. A forward flange 94 is disposed on the forward end of the outer surface of the protrusion 90 and extends beyond the outer perimeter 92 thereof. Finally, a bore is positioned proximate the protrusion 90 spaced apart therefrom toward the distal end 81 of the first temple 24.

The second temple 24′ is shown in FIG. 5. The second temple 24′ is substantially a mirror image of the first temple 24. As such, any similar structures are referred to with the same reference numbers augmented with a prime (′), with the understanding that the structures are mirror images of each other.

It will be understood that the configuration shown substantially precludes relative rotation of the shield 14 vis-a-vis the frame 12 due to the shape of the protrusion 90 and the presence of the bore and nub that interface with the shield 14 (as will be described below). It will further be understood that in other configurations, rotation of the shield 14 may be permitted wherein the protrusion 90 may itself rotate relative to the frame 12 or the protrusion 90 may be shaped so as to allow rotation of the shield 14. That rotation may be indexed through a ratcheting mechanism.

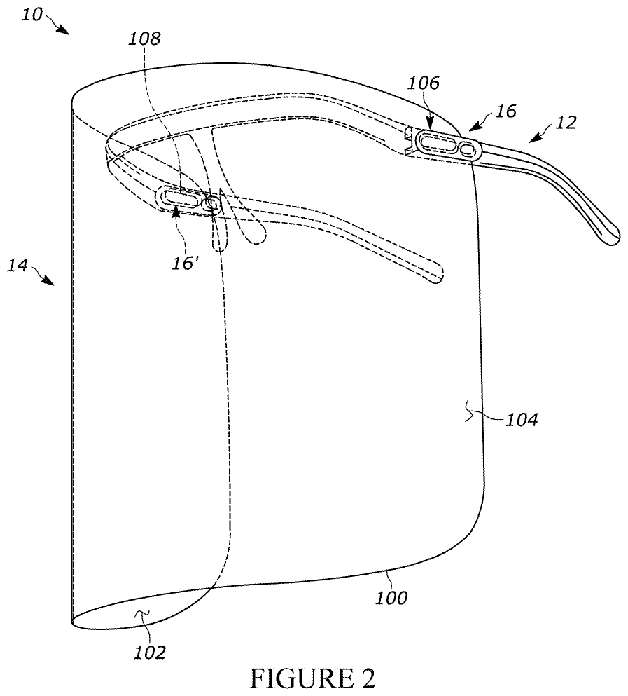

The shield 14 is shown in FIG. 2 as comprising outer perimeter 100, inner surface 102, outer surface 104, first coupling region 106 and second coupling region 108. With reference to FIG. 4, the first coupling region 106 includes bore 110 and indent 112. The bore 110 is inwardly space from the outer perimeter 100 and corresponds to the shape of the protrusion 90 of the first temple 24. The indent 112 forms a portion of the outer perimeter 100 and is configured to curve around the front portion 30 (preferably above and/or below) of bore 96 of the first temple 24. In the example shown, the shield 14 is a full-face shield sized to cover substantially an entire face (not shown) of a wearer of the face shield apparatus 10. It will be understood that the shield 14 may have any number of different configurations, having a full and/or partial facial coverage, similar in size to glasses. The shield 14 can be opaque or tinted, such as to filter out the sun.

Similarly, and with reference to FIG. 5, the second coupling region 108 is a mirror image of the first coupling region 106 and includes bore 114 (that corresponds to the shape of the protrusion 90′ of the second temple 24′) and indent 116 formed as a portion of the outer perimeter 100.

Referring now to FIGS. 10 through 15, the locking clasps 16 (16′ shown in FIG. 2 is substantially identical thereto) are formed from a flexible polymer member that is elastically stretchable and elastically deformable. The first locking clasp 16 is shown as comprising body 120 and interfacing numb 122. The body 120 includes outer surface 130, inner surface 132, front end 134 and back end 136. An indent in the body 120, such as a receiving channel 138 is formed into the inner surface 132 proximate the front end 134. In the example shown, the receiving channel 138 is slightly longer than half a length of the body 120. The receiving channel has a shape that substantially corresponds a shape of the protrusion 90, and includes flange receiver 139 to receive the forward flange 94. The front end 134 of the locking clasp 16 may have an increased thickness to facilitate grasping by a user toward the manipulation of the same.

The interfacing nub 122 includes bore interface 140 and terminates at distal flange 142 (FIG. 10). In the example shown, the bore interface 140 is oval in shape, however, other shapes are possible, such as circular, square, rectangular, or any other shape. In the example shown, the distal flange 142 is oval in shape, however, other shapes are possible, such as circular, square, rectangular, or any other shape. As will be explained below, and with reference to FIGS. 8 and 9, the interfacing nub 122 is configured to extend through bore 96 of the shield attachment member of the first temple 24.

The locking clasp 16′ is substantially identical to that of the locking clasp 16 and utilized in cooperation with the second temple 24′ to retain the shield 14 in cooperation with the second coupling region 108.

In operation, a user is provided with a shield 14 (often in a rolled-up configuration) and the frame 12. The frame 12 may be provided with the locking clasps 16, 16′ coupled thereto. In some configurations, a shipping coupling 300 (such as the shipping coupling shown in FIG. 16) may be provided. The shipping coupling 300 has a body 301, having a notch 302 and an opening 304. The opening 304 is interfaced with the protrusion 90 (FIG. 9) while the notch 302 extends about and interfaces with the interfacing nub 122 (FIG. 8) of the locking clasp 16 (FIG. 8). The shipping coupling mimics a portion of the shield 14 and provides a user with an example of how the shield 14 is to be interfaced with the frame 12.

The user can release the shipping coupling from retention by first deforming the body of the locking clasp so as to decouple the body from the protrusion 90 of the first temple 24. The interfacing nub can remain assembled to the bore of the first temple 24. Once the protrusion 90 is removed from the receiving channel, the shipping coupling can be removed. In other configurations, the shipping coupling may be omitted. Additionally, in some configurations, the user will have to attach the locking clasp to the temples 24 and 24′ by extending the interfacing nub of the locking clasp into the respective bore of each of the first and second temples 24 and 24′.

Once the locking clasps 16, 16′ are in a position wherein they have their interfacing nubs extended through the respective one of the bores of the first and second temple 24 and 24′, the user can unroll the shield 14 (where provided rolled), or if the shield 14 is provided flat, the shield 14 can be immediately ready for attachment. The shield 14 is positioned so that the bore of the first coupling region 106 of the shield 14 is fitted to the protrusion 90. At the same time, the indent of the first coupling region 106 is interfaced with the interfacing nub of the locking clasp.

Once interfaced, the body of the first locking clasp is elastically deformed and extended over the protrusion 90. As the body of the first locking clasp is manipulated, the protrusion 90 is directed into the receiving channel with the forward flange being directed into the flange receiver. Once directed and interfaced, the first locking clasp is released and the shield 14 is sandwiched between the locking clasp and the outer surface of the first temple 24. Rotational movement relative to the frame 12 is precluded due to the configuration of the protrusion 90 and the shape of the bore of the shield 14 so that relative rotational movement is precluded. Additionally, the interface between the interfacing nub and the indent of the shield 14 further aids in precluding relative rotative movement. The shape and dimensions of the locking clasp can be such that the natural resilience of the material of the locking clasp directs a bias against the shield 14 and the outer surface of the frame 12 to further secure the structures together and to prevent relative movement.

Once the first clasp 16 is positioned into locking releasable engagement with the protrusion 90 of the first temple 24, the same process can be repeated with the second coupling region 108 of the shield 14, and, the shield 14 can be interfaced with the protrusion 90 of the second temple 24′, and the second locking clasp. Once both of the locking clasps 16, 16′ are fully installed, the shield 14 is ready for use.

Advantageously, as the user installs the shield 14, it becomes apparent that the top bar 20 of the frame 12 is directed above the eyebrows and into a region of the forehead spaced apart from the eyebrows and the eyes.

In addition, the bridge 22 directs the top bar 20 to be spaced away from the forehead of the user. Further, the bridge 22 is direct inwardly at the nose and can engage the nose very high (at or near the level of the eyes). Such a configuration further places the shield 14 spaced apart from the front portion 30 of the top bar 20 and also away from the face of the user. Due to the placement of the bridge 22 in the orientation described and the position of the front portion 30 of the top bar 20, the user can install glasses such that the bridge 22 is positioned between the bridge 22 of the glasses and the face of the user. Even larger glasses can be accommodated.

Additionally, the first and second end portions 32, 34 wrap around so that the distal ends are directed toward the ears of the user where the first and second temples 24 and 24′ are hingedly joined. Such a configuration moves the coupling of the shield 14 substantially further back and along the sides of the user's head (i.e., out of the way).

Finally, the interface between the shield 14 and the frame 12 is configured to preclude relative rotation between the components, and without having a fastener that extends through the shield 14 and through the frame 12. Rather, the shield 14 has an opening that interfaces with protrusions 90, 90′ in the first and second temple 24 and 24′, and which have resilient locking clasps 16, 16′ that sandwich the shield 14 to the frame 12. Having the protrusions 90, 90′ as part of the temples 24 and 24′ minimizes the relative movement of the shield and the frame 12.

Removal of the shield 14 can easily be achieved through the release of the locking clasps 16, 16′ with the respective protrusions 90, 90′ and forward flange of the first and second temples 24 and 24′. Advantageously, the interfacing nubs remain within respective ones of the bores so that they stay with the frame 12 when the shield 14 is removed. Replacement of a new shield (where a shield has been damaged) or replacement with the same shield can easily be accomplished using the methods described above.

It is contemplated that the first and second temples 24 and 24′ can be collapsed about the hinge 54 while the shield 14 is installed, such that the shield 14 develops a c-shaped configuration.

It is further contemplated that the shield 14 attachment members may comprise a separate component that can be adhered or otherwise coupled to an existing pair of glasses, goggles, or the like. In such a configuration, the shield attachment members comprise a retrofit that with the use of the locking clasps 16, 16′ can releasably attach the shield 14 to a user's existing glasses, goggles or the like.

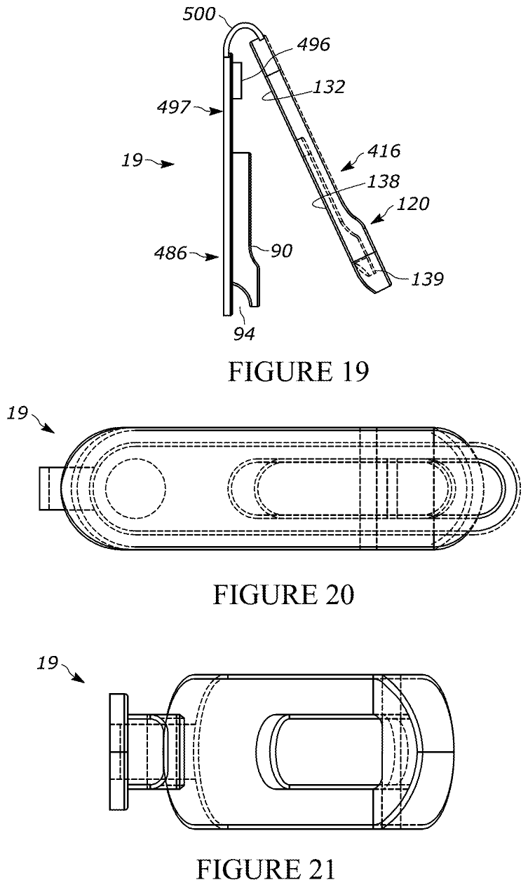

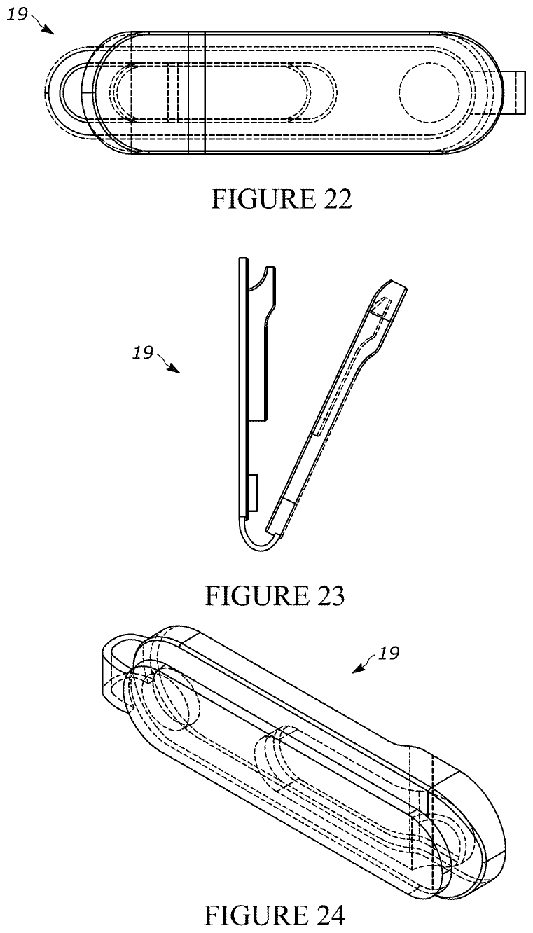

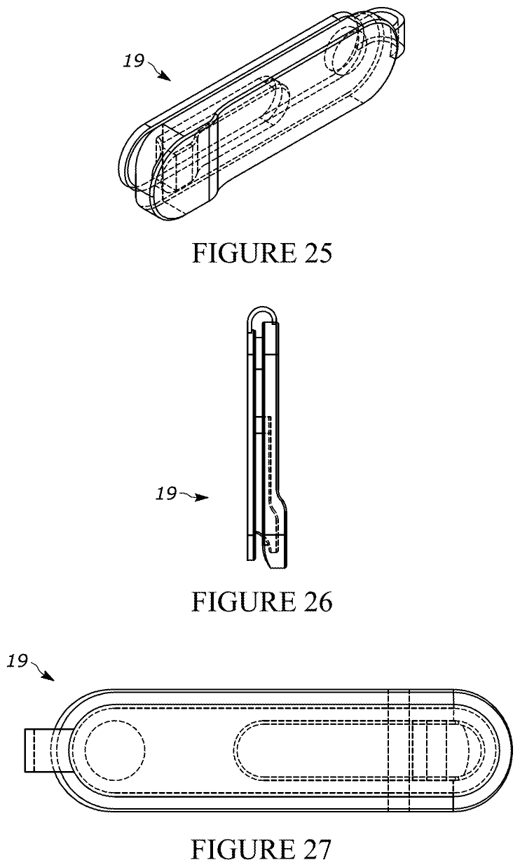

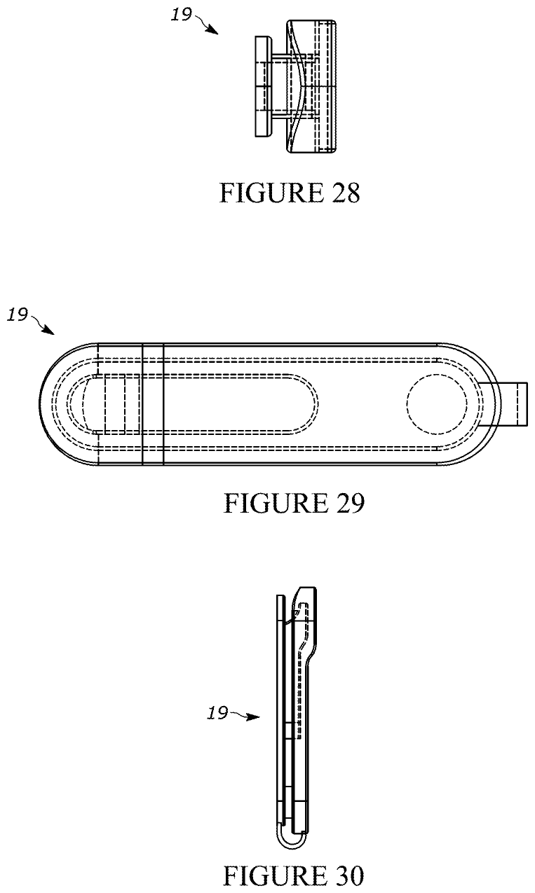

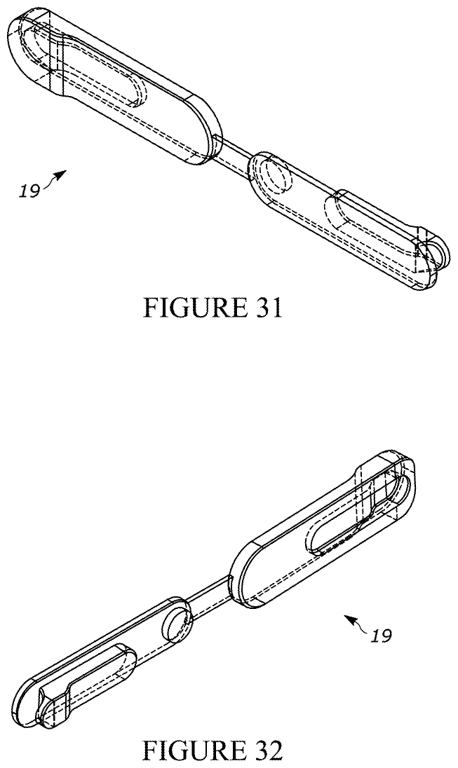

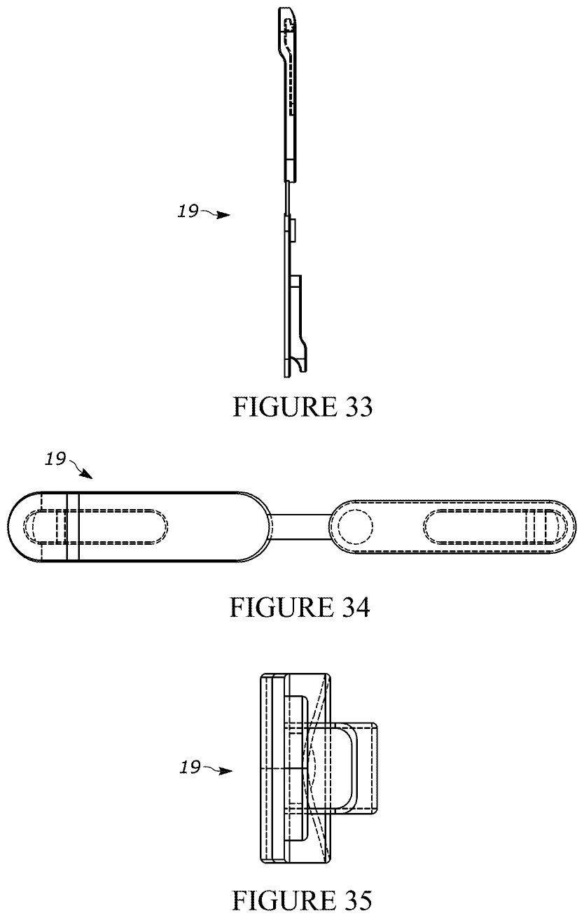

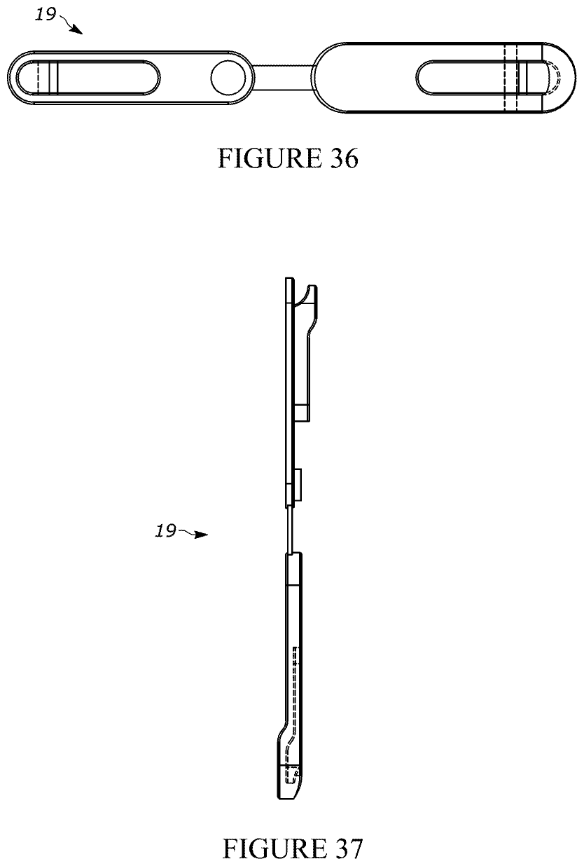

In a configuration which is meant to be illustrative, and not limiting, the standalone shield attachment member 486 with locking clasp 416 (collectively 19) is shown in FIGS. 17 through 37 for fitting onto an existing pair of glasses, goggles or the like. In such a configuration, portions of the shield attachment member are formed on a base 497, such as, for example, the protrusion 90. In at least one configuration, a width of the locking clasp 416 is larger by approximately 20% than a width of the base 497, as shown in FIGS. 17-37, although other widths are possible. The bore 96 can be replaced with nub 496. In at least one example, the nub 496 is circular in shape, as shown, but can be other shapes, such as oval, square, rectangular, or any other shape. The locking clasp 416 can be provided and can be coupled to the base 497 by way of a live hinge 500. In at least one configuration, a width of the live hinge 500 is approximately half a width of the locking clasp 416 and the base 497, as shown in FIGS. 17-37, although other width.

In at least one configuration in which the locking clasp 416 and the base 497 are used to secure the shied 14 to the frame 12, a height of the nub 496 is substantially (+—10%) same as a thickness of the shield 14. This substantially sameness allows the base 497 to be coupled to the locking clasp 416 to thereby form a gap therebetween. This gap is configured to allow the base 497 and the locking clasp 497 to be disposed flush against a surface of the shield 14, as shown in FIGS. 1-7. In such a configuration, the nub 496 is pressed against the inner surface 132 of the body 120 of the locking clasp 416. In some configurations, the nub 496 may press against a flat surface, or may press against an indented surface. The entire member can be attached to an existing pair of glasses, goggles or the like through adhesive or through other structures such as elastomeric sleeves or the like. Typically, the base 497 is attached to the temple of the existing pair of glasses, goggles or the like. The closing of the clasp 19 and the opening thereof is similar in operation to that which is described above. It will be understood that the nub 496 may be varied, as can the structure of the protrusion 90, as well as the configuration of the live hinge 500. In other configurations, the locking clasp 416 may be a separate member and not coupled to the base 497 with the live hinge 500.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

What is claimed is:

1. A pair of opposed locking clasps to couple a temple of a frame to a top bar of the frame, the pair of opposed locking clasps comprising:
a first locking clasp comprised of a first base, a nub disposed proximate to a first end of the first base, and a protrusion disposed proximate to a second end of the first base;
a second locking clasp comprised of a second body and an indent that substantially corresponds to a shape of the protrusion, the protrusion to be disposed in the indent to lock the first locking clasp to the second locking clasp; and
a hinge to couple the first locking clasp to the second locking clasp.

2. The pair of opposed locking clasps according to claim 1, wherein the protrusion includes a non-uniform outer perimeter so as to preclude rotative movement therearound.

3. The pair of opposed locking clasps according to claim 1, wherein the protrusion is an elongated racetrack oval.

4. The pair of opposed locking clasps according to claim 1, wherein the protrusion includes a forward flange disposed on a forward end of the protrusion and extends beyond an outer perimeter of the protrusion.

5. The pair of opposed locking clasps according to claim 1, wherein the hinge is a live hinge.

6. The pair of opposed locking clasps according to claim 1, wherein the nub is circular in shape.

7. The pair of opposed locking clasps according to claim 1, wherein the pair of opposed locking clasps comprise a flexible polymer.

8. The pair of opposed locking clasps according to claim 1, wherein the frame is molded from a polymer.

9. The pair of opposed locking clasps according to claim 8, wherein the polymer is at least one of TR90 and a nylon.

10. A face shield apparatus comprised of the pair of opposed locking clasps according to claim 1, wherein the face shield apparatus further comprises a shield that is coupled to the fame via the pair of opposed locking clasps.

11. The face shield apparatus according to claim 10, wherein the shield is a full-face shield.

12. The face shield apparatus according to claim 10, wherein the shield wraps around a face of a wearer of the face shield apparatus.

13. A face shield apparatus comprising:
a pair of opposed locking clasps to couple a temple of a frame to a top bar of the frame, the pair of opposed locking clasps comprising:
a first locking clasp comprised of a first base, a nub disposed proximate to a first end of the first base and a protrusion disposed proximate to a second end of the first base;
a second locking clasp comprised of a second body and an indent that substantially corresponds to a shape of the protrusion, the protrusion to be disposed in the indent to lock the first locking clasp to the second locking clasp; and
a hinge to the first locking clasp to the second locking clasp;
a shield to couple to the fame via the pair of opposed locking clasps.

14. The face shield apparatus according to claim 13, wherein the protrusion includes a non-uniform outer perimeter so as to preclude rotative movement therearound.

15. The face shield apparatus according to claim 13, wherein the protrusion is an elongated racetrack oval.

16. The face shield apparatus according to claim 13, wherein the protrusion includes a forward flange disposed on a forward end of the protrusion and extends beyond an outer perimeter of the protrusion.

17. The face shield apparatus according to claim 13, wherein the hinge is a live hinge.

18. The face shield apparatus according to claim 13, wherein the nub is circular in shape.

19. The face shield apparatus according to claim 13, wherein the pair of opposed locking clasps comprise a flexible polymer.

20. The face shield apparatus according to claim 13, wherein the shield is a full-face shield.
